(19) United States
(12) Patent Application Publication
(10) Pub. No.: US 2015/0150482 A1
(43) Pub. Date: Jun. 4, 2015

(54) METHOD AND SYSTEM FOR IMAGING

(71) Applicant: MONASH UNIVERSITY, Clayton, Victoria (AU)

(72) Inventors: Andreas Fouras, Park Orchards (AU); Jordan Thurgood, Parkdale (AU)

(73) Assignee: MONASH UNIVERSITY, Clayton, Victoria (AU)

(21) Appl. No.: 14/395,086

(22) PCT Filed: Apr. 16, 2013
(86) PCT No.: PCT/AU2013/000390

§ 371 Date: Oct. 17, 2014

(30) Foreign Application Priority Data
Apr. 17, 2012 (AU) 2012901499

(51) Int. Cl. A61B 5/08; A61B 6/03; A61G 10/02; A61B 5/00; A61M 16/00; A61B 5/055; A61B 8/08

(52) U.S. Cl. CPC A61B 5/08; A61B 5/055; A61B 6/032; A61B 8/483; A61B 5/742; A61M 16/0057; A61M 16/0006; A61M 16/0096; A61G 10/026

(57) ABSTRACT

A method for dynamic investigation of a subject lung, the method comprising the steps of: (i) imparting an oscillation to the lung at one or more forcing frequencies so as to elicit a lung response, (ii) sensing the response of the lung simultaneously with the imparting of the oscillation to elicit a lung response, (iii) choosing at least one parameter used in the sensing to define the lung motion associated with the lung response, (iv) comparing one of the chosen parameters at each forcing frequency with the response at the forcing frequency in at least one region of the lung, and (v) recording the comparison of step (iv).

The present invention relates to the field of imaging for physiological, clinical or research applications.

In one form, the invention relates to dynamic lung function measurement in a human or animal.

In one particular aspect the present invention is suitable for use in lung function testing for assessing lung function and lung condition.

### BACKGROUND ART

It is to be appreciated that any discussion of documents, devices, acts or knowledge in this specification is included to explain the context of the present invention. Further, the discussion throughout this specification comes about due to the realisation of the inventor and/or the identification of certain related art problems by the inventor. Moreover, any discussion of material such as documents, devices, acts or knowledge in this specification is included to explain the context of the invention in terms of the inventor's knowledge and experience and, accordingly, any such discussion should not be taken as an admission that any of the material forms part of the prior art base or the common general knowledge in the relevant art in Australia, or elsewhere, on or before the priority date of the disclosure and claims herein.

Lung diseases adversely affect airflow during breathing and alter normal lung motion. Specifically, lung diseases change the elasto-mechanical and aero-resistive properties of the lung which in turn alters the airflow in and out of the lung. For example, interstitial fibrosis increases distal airway stiffness, asthma increases airway resistance and emphysema reduces lung tissue recoil thereby increasing its compliance. Although these diseases differ markedly in both cause and consequence, they all alter the mechanical properties of diseased regions and therefore must also alter motion of these regions.

Little is known about the dynamics of lung motion during respiration, particularly how different regions of the lung move in relation to other regions during both inspiration and expiration. It is not known whether the lung expands and deflates uniformly, or whether specific regions lead or trail other regions due to differences in local compliances or proximity to the diaphragm. Similarly, it is unknown how diseases affect regional lung motion and whether motion in healthy regions is altered to compensate for diseased regions. Although this information is best provided by imaging the lung in situ, it has not hitherto been possible to image the lung with sufficient spatial and temporal resolution.

Previous techniques to measure lung motion have relied on the surgical placement of markers, inhalation of contrast agents or removal of the chest wall for imaging.

Forced Oscillation Technique (FOT) is a very popular and successful global lung function test. FOT works by applying an oscillation to the airway opening and then simultaneously measuring the pressure and flow at the airway opening. FOT determines the impedance of the lungs on a global basis. This technique is popular for determining the state and function of lung tissue non-invasively by measuring the lungs' reaction to a series of input oscillations. Oscillations are generally in the order of 4-48 Hz and as a result any technique to measure the lung response across such a broad range will obviously require very high temporal resolution. For example, U.S. Pat. No. 5,318,038 (Jackson et al) describes an infant respiratory impedance measuring apparatus and method that use FOT.

One of the drawbacks of this technology is that it cannot measure response locally with in the lungs and as a result it is unable to detect any but the largest physiological changes in the lungs. Due to the global nature of FOT and the potential of destructive interference between different signals in lung regions there is the likelihood that this approach will result in lost information.

Standard imaging techniques such as X-ray Computer Tomography (CT) and Magnetic Resonance Imaging (MRI) imaging during breath-holds provide little or no information on lung motion and cannot detect disease that cause subtle changes in lung structure. These approaches are particularly limited by the need to image the lung while it is stationary to minimise blurring. In particular, MRI and CT have poor temporal resolution preventing them from being used to image the lungs during a dynamic lung test. Furthermore, due to long acquisition times, both MRI and CT are often used to compare the state of the lung at two different time intervals, usually minutes apart. Interpolation is required to deduce lung motion between two steady state conditions within a breath and such methods assume that the motion follows a linear or defined path. This has obvious drawbacks and limits the ability of the techniques to be used for dynamic lung function testing.

Clinical gated 4D-CT has also been used for measurement of lung function, including expansion using traditional absorption based imaging at the expense of significant levels of radiation dose. Typically, the phase matching is performed to an accuracy of 7.1% of the breath cycle or 400 ms. This results in poor temporal resolution for investigation for the dynamic patterns of motion and expansion within the lung, particularly for small animal studies.

Vibration Response Imaging (VRI) is a technology developed for investigating regional lung function and for diagnosis of conditions. US patent application 2007/0244401 relates to a method and system for assessing an interventional pulmonology procedure including VRI imaging. Images indicative of airflow in at least a portion of the respiratory tract are generated from signals indicative of pressure waves at transducers applied to the skin of a subject. Specifically, the signals are measured before and after the interventional pulmonology procedure and used to generate images for comparison. This technique however, suffers from very poor spatial resolution and is based on measurements taken through the chest wall resulting in poor dynamic range of measurements.

Electrical Impedance Tomography (EIT) is a technique of measuring the impedance through the chest of an electrical signal. EIT provides information through a horizontal slice of the lung, is easily affected by the electrical signals of the heart and has very poor spatial resolution. Typically temporal resolutions of only up to 44 Hz have been used.

Despite advances in lung imaging, altered patterns of lung motion have not hitherto been utilised for disease detection. Accordingly, there is a need for improved technologies for assessing lung function and diagnosing lung conditions.

### SUMMARY OF INVENTION

An object of the present invention is to provide improved technology for assessing lung function and diagnosing lung conditions.

Another object of the present invention is to provide an improved method for dynamic lung function testing.

Another object of the present invention is to provide improved technology for assessing lung function and lung condition in a localised manner.

Another object of the present invention is to provide a method and apparatus for measuring regional respiratory impedance using forced oscillations.

A further object of the present invention is to alleviate at least one disadvantage associated with the related art.

It is an object of the embodiments described herein to overcome or alleviate at least one of the above noted drawbacks of related art systems or to at least provide a useful alternative to related art systems.

In a first aspect of embodiments described herein there is provided a method for dynamic investigation of a subject lung, the method comprising the steps of:
(i) imparting an oscillation to the lung at one or more forcing frequencies so as to elicit a lung response,
(ii) sensing the response of the lung simultaneously with the imparting of the oscillation to elicit a lung response,
(iii) choosing at least one parameter used in the sensing to define the lung motion associated with the lung response,
(iv) comparing one of the chosen parameters at each forcing frequency with the response at the forcing frequency in at least one region of the lung, and
(v) recording the comparison of step (iv).

Typically the sensing will comprise imaging the response of the lung. Any form of internal or external oscillation may be imparted to the lung—including mechanical input oscillation, an external chest wall oscillation, the heart's oscillation or any other internal bodily oscillation or any other externally applied oscillation.

Typically the dynamic investigation comprises measurement of respiratory impedance, tissue elasticity or other tissue properties, or any other mechanisms for energy transfer in at least one region of the subject lung.

The combination of any method of lung imaging with the established ideas of FOT has the potential to provide information provided by FOT but in individual or multiple regions of the image. Established lung imaging systems typically provide at least 2,000 to 1,000,000 or more measures of the lung. This means the combined technique of imaging FOT has an increased richness of up to 1,000,000 times over FOT. Traditional FOT has the drawback in that it is a global measure and as a result is only suited to detecting large physiological changes in lungs. For example, a large physiological change could comprise a moderate change to tissue properties affecting a large portion of the lung. More subtle changes to tissue properties would only be detectable by traditional FOT if they affected a larger portion of the lung, and changes affecting smaller portions of the lung would only be detectable by traditional FOT if they had resulted in a larger affect on local tissue properties.

In addition FOT testing has the potential to suffer from destructive interference wherein one region of the lung compensates for deterioration in other regions of the lung. More specifically, phase shifts of the forcing frequency can be induced by damping properties of the lungs. Destructive interference is the phenomenon whereby waves that are out of phase cancel each other out and hence this information is lost without any capacity to understand that this has happened. In this situation a global measure would not be sufficient for the detection for the localised deterioration in lung state and/or function.

The method of the present invention may comprise the additional step (vi) of comparing a parameter between different regions throughout the lung and creating a visual representation thereof.

The lung responds to an oscillating input to the airway opening via movement of the lung tissue. This movement can potentially reveal detailed information regarding the state and function of the lungs. In the present invention, the oscillation provided to the lung may be of a single frequency, but typically multiple frequencies will be used. The frequencies could be provided simultaneously or one after the other. Oscillations can either be measured at many images per cycle of lowest frequency or an ensemble type average recorded over many cycles, therefore capable of being recorded at a slower rate than the input oscillations.

Imaging the motion of the lung may be carried out by any suitable method in 1D (single point in the lung), 2D or more preferably 3D, such as MRI, CT, X-ray imaging, and ultrasound or any other suitable form of imaging. A particularly preferred imaging method is phase-contrast x-ray imaging (CTX) as described in Australian patent application AU-2009/904481. Phase-contrast x-ray imaging provides images of high contrast and spatial resolution with temporal resolutions that allow multiple images to be acquired throughout the respiratory cycle. Thus, coupling x-ray phase contrast imaging with velocimetry can be used to measure lung tissue movement and determine velocity fields that define speed and direction of regional lung motion throughout a breath of a human or animal subject.

Movement of lung tissue shows the state of the lung tissue and enables regional measures. Air flow and breathing behaviours can be deduced from these measures. The parameter used for comparison may be any convenient parameter such as relative power, phase or amplitude at each forcing frequency. For example, it is possible to image lung motion of an input oscillation using phase contrast x-ray images of the lungs. Suitable measures extracted could include, for example (a) the frequencies of the oscillatory response of the lung tissued (response oscillation) oscillation, (b) phases of the response oscillations or (c) qualitative measures of strength of response oscillations at each frequency.

Regional maps of lung tissue motion reveal both the heterogeneity of normal lung motion, as well as any abnormal motion.

Other comparisons (and contrasts) may provide valuable information, such as comparisons of low frequency results against high frequency, or comparisons relating to phase, timing, regions, amplitudes.

This is typically carried out by the use of the Fourier Transform or other similar mathematical transformation.

In another aspect of embodiments described herein there is provided an apparatus for dynamic investigation of a subject lung using the method of the present invention, wherein the apparatus comprises:
a ventilator for delivering fluid pressure to the lung;
a means for imparting an oscillation to the lung at one or more forcing frequencies so as to elicit a lung response
a means of sensing the motion of the lung simultaneously with imparting of the oscillation to elicit a lung response;
a means for measuring at least one parameter used in sensing and associated with the lung response at the forcing frequency in at least one region of the lung;
processing means for comparing one of the sensing parameters at the forcing frequency with the response at the forcing frequency in at least one region of the lung;
a means for recording the results of the comparison for at least one region of the lung.

Preferably the sensing parameter is a parameter used in imaging.

Clearly it is preferable for the method of the present invention to be carried out using a ventilator that can maintain stable and accurate pressure to the subject while the oscillation is delivered to the lung as well as being synchronised with imaging equipment and other devices such as data acquisition or medical equipment. However, many of the ventilators of the prior art cannot maintain sufficiently accurate pressure or timing. It is particularly preferable for the method of the present invention to be carried out using a ventilator that can provide high frequency ventilation (HFV) and/or provide the forced oscillation. HFV uses low tidal volumes at high rates to oscillate air into the subject and keeps the lung continuously inflated. Air mixing occurs by various mechanisms including direct bulk flow, Taylor dispersion, Pendelluft flow, cardiogenic mixing and molecular diffusion.

In a preferred embodiment, the ventilator for delivering fluid pressure to the lungs of a subject has a pump in operative connection with a first pressure vessel for control of the peak inspiratory pressure (PIP) of the subject wherein the volume of the first pressure vessel is substantially greater than the volume of the lungs of the subject. It is noted however that by using a sufficiently high flow rate pump and sufficiently fast acting valves and feedback system, increasingly smaller pressure vessels may be used.

The pressure in the ventilator may be controlled by any convenient means. For example, in one embodiment the pressure may be controlled through a sequence of measurements (via sensors) and adjustment of valves controlled through a computer interface and software. In another more preferred embodiment, the ventilator pressure may be controlled through a feedback sequence, which in turn is controlled locally by a microprocessor within the ventilator. The latter embodiment provides a much faster and more stable system.

In a further aspect, the ventilator for delivering fluid pressure to the subject lung has:
a pump in operative connection with a first pressure vessel for control of the peak inspiratory pressure (PIP) of the subject, and
a housing for enclosure of the subject, the housing being in operative connection with the first pressure vessel.

In a particularly preferred embodiment the ventilator has at least two chambers, and is capable of performing HFV. In a yet further embodiment the ventilator has three chambers and is capable of performing HFV. In one embodiment, the ventilator vents to the atmosphere. In another embodiment the ventilator includes a second pressure vessel for control of the positive end expiratory pressure (PEEP).

In a yet further aspect, the ventilator for delivering fluid pressure to a subject has:

a pump in operative connection with a first pressure vessel for control of the peak inspiratory pressure (PIP) of the subject,

a second pressure vessel for control of the positive end expiratory pressure (PEEP) of the subject,

a housing for enclosure of the subject, the housing being in operative connection with the first pressure vessel and the second pressure vessel,

wherein the volumes of the first pressure vessel and the second pressure vessel are substantially greater than the volume of the lungs of the subject.

During ventilation of a subject, air flows from the first pressure vessel into the housing as the subject's lungs are inflated (inspiration); air flows from the housing into the second pressure vessel (expiration) and the subject's lungs are deflated.

In another aspect of embodiments described herein stable pressure is delivered to the lungs of a subject, the method of the present invention includes the steps of:
(1) enclosing the subject within a housing, the housing being in operative connection with a first pressure vessel held at a first (PIP) pressure and a second pressure vessel held at a second (PEEP) pressure, respectively,
(2) admitting air from the first pressure vessel into the housing, then
(3) admitting air from the housing into the second pressure vessel, and
(4) repeating steps (2) and (3) multiple times.

If the inspiration time is sufficiently long, or the flow rate is sufficiently fast, the air may be admitted until the housing reaches a desired first pressure (in step 2) or a second pressure (in step 3).

Preferably the pressure within the vessels does not change by more than ±10%, preferably not more than ±5%, even more preferably not more than ±1%.

The pressure can be maintained through the use of a pressure vessel substantially larger than the inspired or expired volume. Typically the pressure vessels are each at least 10× the inspired or expired volume, more preferably at least 100× the inspired or expired volume.

Alternatively the pressure can be maintained by the feedback control system. As the feedback control system performance is increased, smaller pressure vessels will be required.

Other aspects and preferred forms are disclosed in the specification and/or defined in the appended claims, forming a part of the description of the invention.

In essence, embodiments of the present invention stem from the realization that conventional spirometry techniques such as FOT can be combined with lung imaging to provide information specific to every individual region across the image. Specifically they can be used to measure feature such as lung tissue movement and to determine velocity fields that define speed and direction of regional lung motion throughout a breath.

Advantages provided by the present invention comprise the following:
improved spatial and temporal resolution for assessing lung function and diagnosing lung conditions;
improved assessment and diagnosis of the lung in a localised matter;
accurate measures of regional lung function;
improved dynamic lung function testing;
improved identification of subtle changes of structure in (such as during the early stages of a disease or other disorder);
synchronisation between ventilation and image acquisition to facilitate collection of data; and
simultaneous lung function testing during ventilation

Further scope of applicability of embodiments of the present invention will become apparent from the detailed description given hereinafter. However, it should be understood that the detailed description and specific examples, while indicating preferred embodiments of the invention, are given by way of illustration only, since various changes and modifications within the spirit and scope of the disclosure herein will become apparent to those skilled in the art from this detailed description.

### BRIEF DESCRIPTION OF THE DRAWINGS

Further disclosure, objects, advantages and aspects of preferred and other embodiments of the present application may be better understood by those skilled in the relevant art by reference to the following description of embodiments taken in conjunction with the accompanying drawings, which are given by way of illustration only, and thus are not limitative of the disclosure herein, and in which:

FIG. 1(a) is a plot of input pressure at the airway opening of a subject against time to illustrate input pressure oscillation. FIG. 1(b) is a plot of the vector divergence against time to illustrate the measured vector divergence;

FIG. 2(a) is a plot of frequency response of the pressure input from FIG. 1, in the power spectrum to illustrate input pressure oscillation.

FIG. 2(b) is a plot of the frequency response of the measured vector divergence of FIG. 1;

FIG. 3 illustrates lung maps showing lung expansion at specific frequencies, the broken line indicating heart oscillation, the unbroken line indicating input oscillation;

FIG. 4(a) and FIG. 4(b) are plots of amplitude versus frequency of the measured response after Fourier transformation of the global average (FIG. 4(a)) and the individual vectors (FIG. 4(b)) to illustrate the horizontal (3) and vertical (1) velocity components;

FIG. 5 is an image of subject lungs generated using the method of the present invention using an amplitude of oscillation of 4 Hz with the subject lying on its side;

FIG. 6 is a plot of power versus frequency before (circles, 5) and after (squares, 7) the delivery of a dose of aerosol methacholine. There is a measurable decrease in the global average of expansion after the delivery of methacholine;

FIG. 7(a) and FIG. 7(b) correspond to FIG. 6 and are power maps of the 4 Hz oscillations before the delivery of methacholine (FIG. 7(a)) and post delivery of methacholine (FIG. 7(b));

FIG. 8(a) is a schematic diagram of an imaging configuration according the present invention; FIGS. 8(b) and 8(c) illustrate the contrast for lung tissue obtained through phase-contrast x-ray imaging over absorption-based x-ray imaging. The various components included in the apparatus for imaging depicted in FIG. 8(a) are as follows:

synchrotron storage ring, 9

bonding magnet, 11

monochromators, 13

x-ray beam, 15

sample, 17

scintillator, 19

optical lens, 21

optical mirror, 23

detector, 25; and

phase contrast image, 27;

FIG. 9(a) shows the 3D nature of x-ray illumination and velocimetric cross-correlation analysis; FIG. 9(b) shows in vivo detection of lung tissue motion. The various components depicted in FIG. 9(a) are as follows;

X-ray beam, 29

lungs, 31

lung volume, 33

projection images at t₁ 35a, and t₂, 35b,

graph of velocity distribution, 37

cross-correlation, 39;

FIG. 10 shows the empirical relationship between lung divergence and tissue expansion; FIG. 10(a) is a graph of integrated divergence (px) against lung volume (mL) where Int.Div=227.64×lung volume, and R2=0.98; and FIG. 10(b) is a graph of lung volume (mL) against time measured using a plethysmograph (circles, 41) with normalised integrated divergence (crosses, 43);

FIG. 11 shows physiological measures of lung pathology comparing controls with groups 36 hours after bleomycin exposure (FIGS. 11(a) & (b)) and 6 days after exposure (FIGS. 11(c) & (d). In each graph the control result is depicted in black and the bleomycin result is depicted in white. In FIG. 11(b) the bleomycin p<0.001. In FIG. 11(d) the bleomycin p=0.02;

FIG. 12 shows velocimetric measures of lung pathology comparing controls with groups 36 hours (FIG. 12(a) and 6 days (FIG. 12(b)) after bleomycin exposure. In each graph the control result is depicted by the black circles (47) and the bleomycin at p, 0.001 by the white circles (49);

FIGS. 13(a) and 13(b) illustrate regional divergence with the lung and matching histology in FIGS. 13(c), 13(d) and 13(e);

FIG. 14 is a schematic representation of the electrical wiring between components of one embodiment of a ventilator and data acquisition system suitable for use in the method and apparatus of the present invention, wherein black lines represent outputs and grey lines represent inputs from the data acquisition system. The components are as follows,

Personal computer, 51

Data acquisition box, 53

Peak inspiratory pressure (PIP) differential pressure transducer (DPT), 55

PIP valve, 57 (venting to atmosphere)

PIP vessel, 59

Positive end-expiratory pressure (PEEP) vessel, 61

Inspiration solenoid valve, 63

Expiration solenoid valve, 65

Inspiration valve, 67

Expiration valve, 69

PEEP DPT, 71

PEEP valve, 73 (venting to atmosphere)

Airway pressure DPT, 75;

FIG. 15 is a schematic representation of air flow through the ventilator of FIG. 14, with arrows indicating the direction of flow which is generated by a gas pump (77) through a muffler (79) in relation to a subject lung (81).

### DETAILED DESCRIPTION

Although many different imaging methods are suitable for use in the present invention, in a particularly preferred embodiment the present invention uses phase-contrast x-ray imaging (PCXI). PCXI exploits the phase change caused by x-ray refraction when passing between media of differing refractive indices to produce high contrast images of the lung. Interference between transmitted and refracted x-rays produces high contrast images of the air/tissue boundaries compared with conventional x-ray absorption techniques. It is able to achieve this because the phase shift of the x-rays is generally more than three orders of magnitude greater than the absorption over the diagnostic x-ray energy range (20 keV-90 keV). An important consequence of this is that phase-contrast images can be recorded with significantly lower dose than conventional images, which is particularly important for both longitudinal studies and dynamic studies where repeated imaging is required. These benefits are particularly relevant to scientific use. For clinical use, reduced radiation dose is of value for reduction in cancer risk.

Since lung tissue motion is complex, dynamic and heterogeneous, the present invention is a novel application of the mathematical concept of divergence. In particular, even when the imaging used is of a two-dimensional nature, the divergence measure is highly correlated to changes in lung volume.

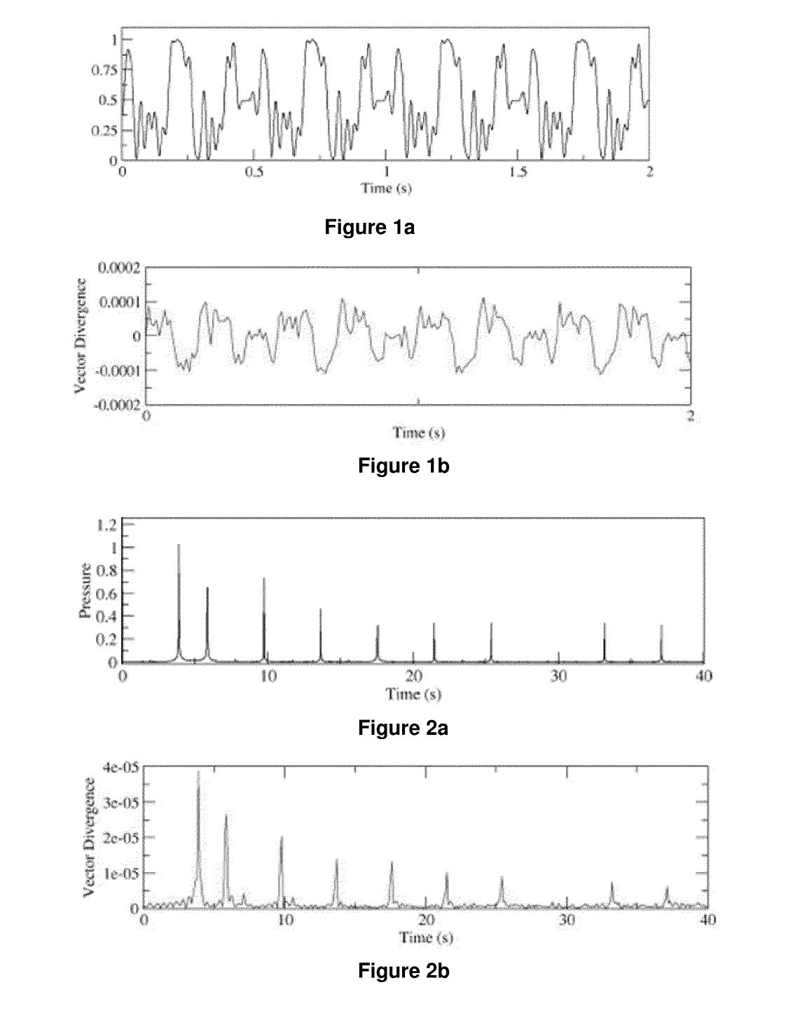

FIG. 1(a) illustrates a plot of input pressure perturbations as measured with a pressure sensor at the airway opening. The input signal is composed of 9 distinct and different frequencies. FIG. 1(b) illustrates a plot of the vector divergence measured via a cross-correlation technique on phase contrast X-ray lung images. This is the global average of all locations across the lung (>1000 locations). Each vector location produces its own response to the input pressure wave, correlating to the specific local lung properties in the region.

FIG. 2(a) illustrates frequency response of the pressure input from FIG. 1, in the power spectrum. FIG. 2(b) illustrates frequency response of the measured vector divergence of FIG. 1. The 9 distinct frequencies can be obtained from the response of either pressure at the airway opening or the global average of vector divergence.

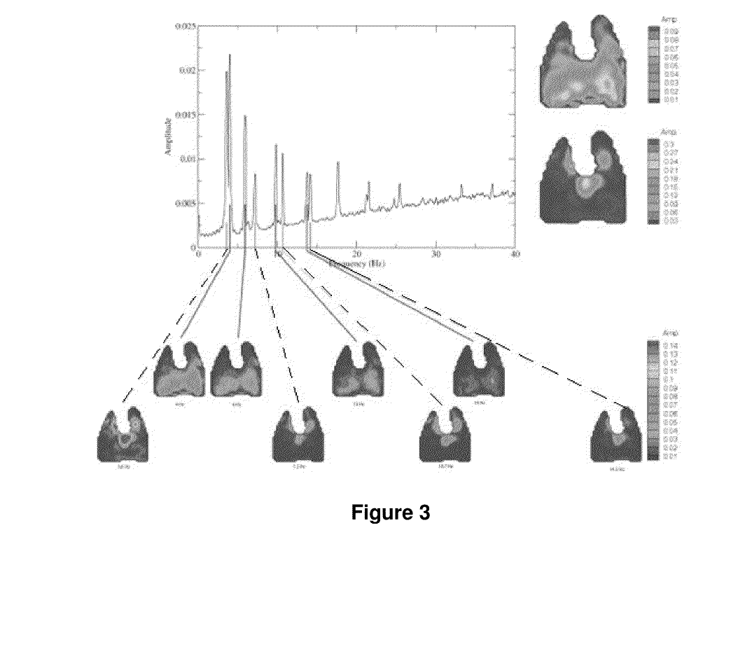

FIG. 3 illustrates lung maps showing lung expansion at specific frequencies. Both the heart and the pressure inputs contribute to lung expansion at different frequencies, the heart's first harmonic being at 3.6 Hz. The pressure wave input frequencies are 4, 6, 10 and 14 Hz. The heart harmonics can be seen and measured at 3.6, 7.2, 10.7 and 14.2 Hz. Above right is a larger version of the 4 Hz oscillation to highlight the distribution of power of oscillations at that specific frequency. In the contour maps the large amount of information obtained from this technique is shown as each vector location (>100 across the lung) provides its own complete measure of local lung health.

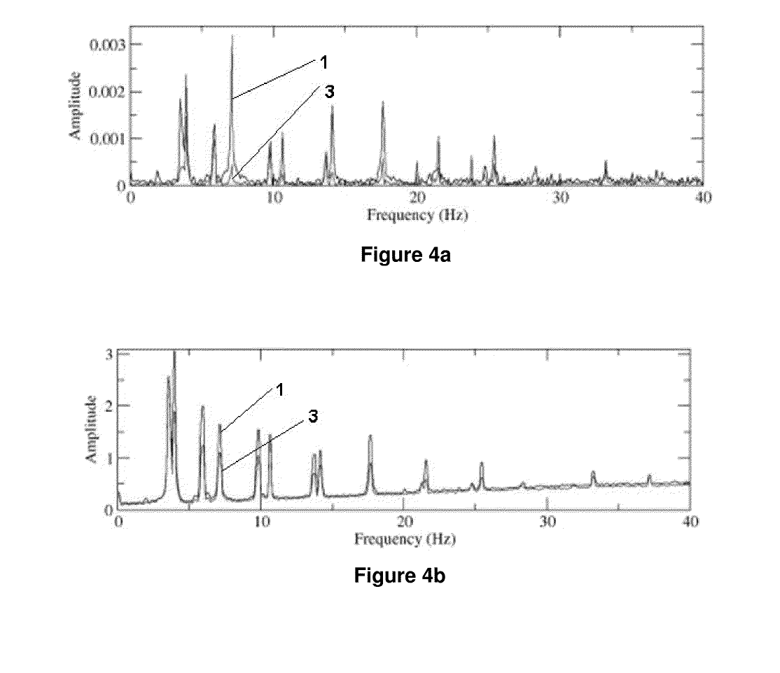

FIG. 4 illustrates the U and V velocity components measured as (a) an fft of the global average of lung expansion or (b) an fft of each vector then globally averaged. The first method more clearly highlights the difference between U and V velocity components that are created by the heart. This can be used to identify the frequencies at which the heart has an effect as well as their relative magnitudes, thus allowing for very accurate filtering of the heart.

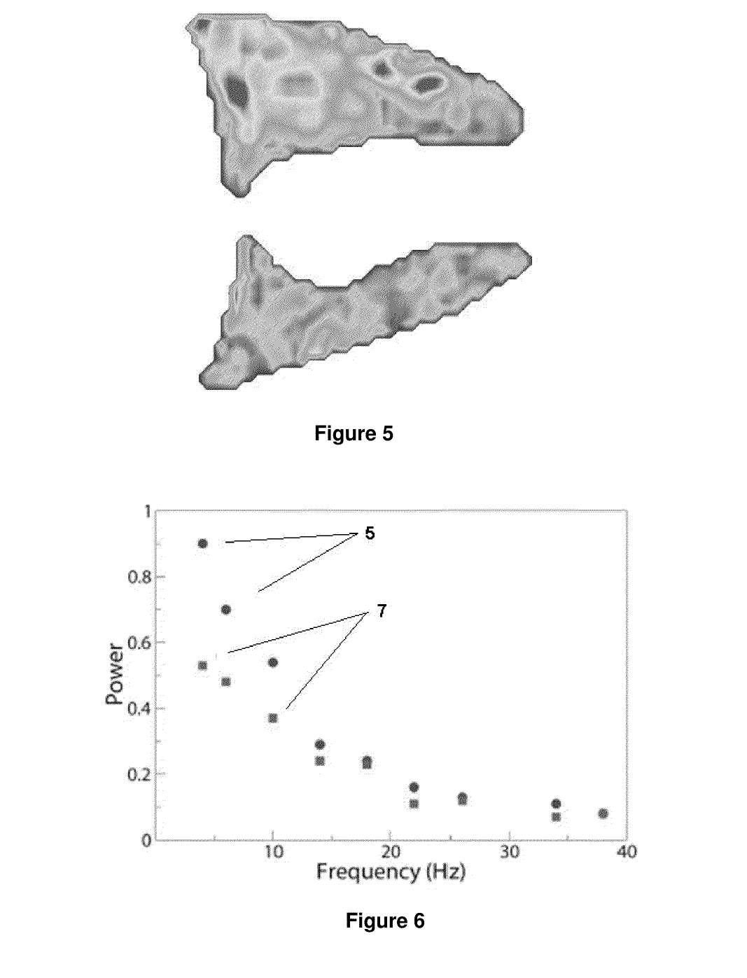

FIG. 5 illustrates amplitude of oscillation at 4 Hz with the subject lying on its side. Note that the bottom lung is supporting the weight of the other lung and the heart above it, and as a result has less measured oscillation amplitude. It appears that the more inflated the lung, the greater the oscillation amplitude. This technique is thereby suitable to measure regional affects for not only lung health but also lung mechanics and posture related changes.

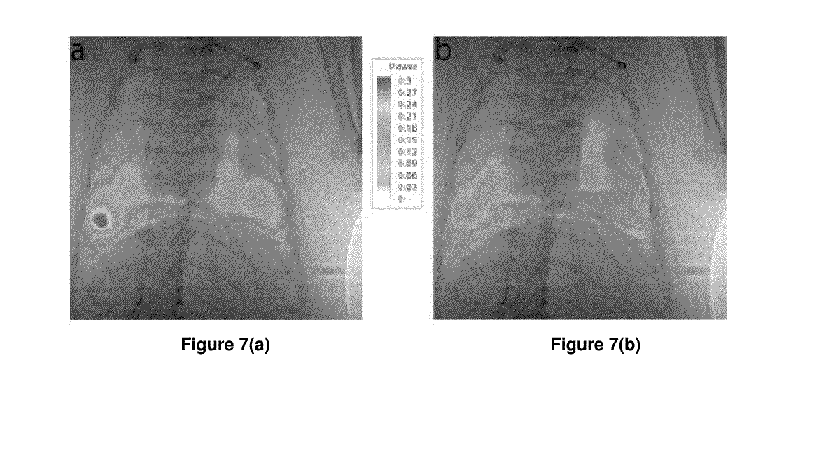

FIG. 6 illustrates the power of input oscillations before (blue) and after the delivery of a dose of aerosol methacholine (red). There is a measurable decrease in the global average of expansion after the delivery of methacholine. FIG. 7(a) and FIG. 7(b) illustrates corresponding power maps of 4 Hz oscillations before the delivery of methacholine (FIG. 7(a)) and post delivery of methacholine (FIG. 7(b)).

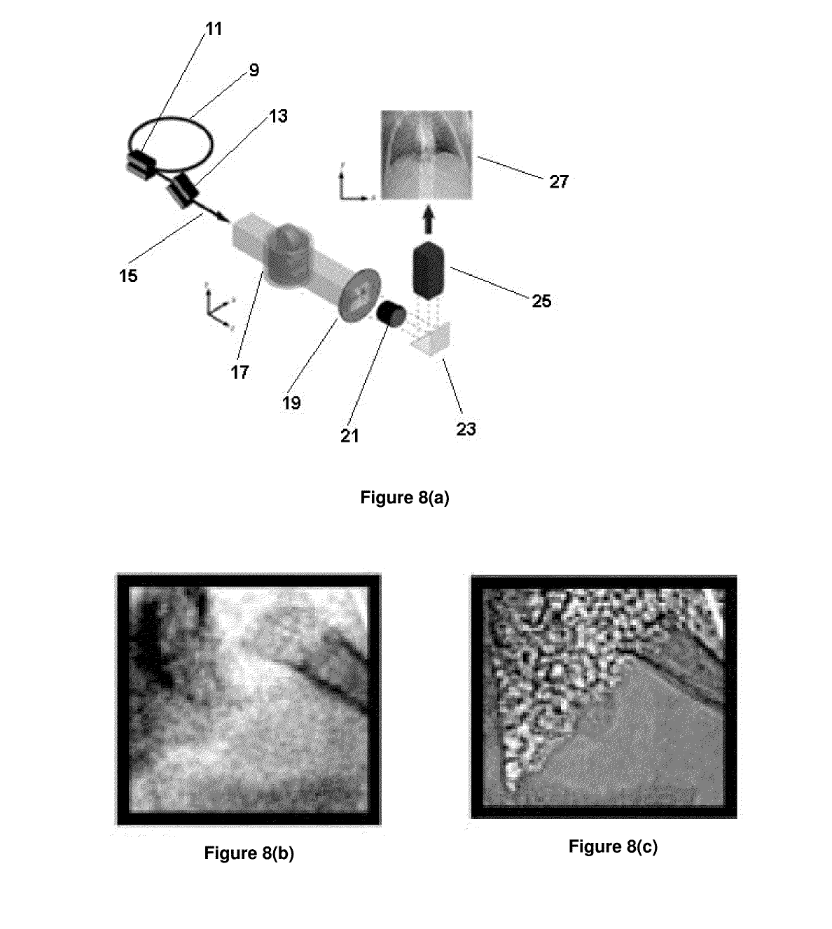

FIG. 8(a) is a schematic diagram showing a suitable configuration for Phase-contrast x-ray imaging. FIGS. 8(b) and 8(c) are examples of the possible increase in contrast for lung tissue obtained through phase-contrast x-ray imaging over absorption-based x-ray imaging. PXCI exploits the phase change caused by x-ray refraction when passing between media of different refractive indices to produce high contrast images of the lung. Interference between transmitted and refracted x-rays produces high contrast images of the air/tissue boundaries compared with conventional x-ray absorption techniques as illustrated by the images shown at FIGS. 8(b) and 8(c).

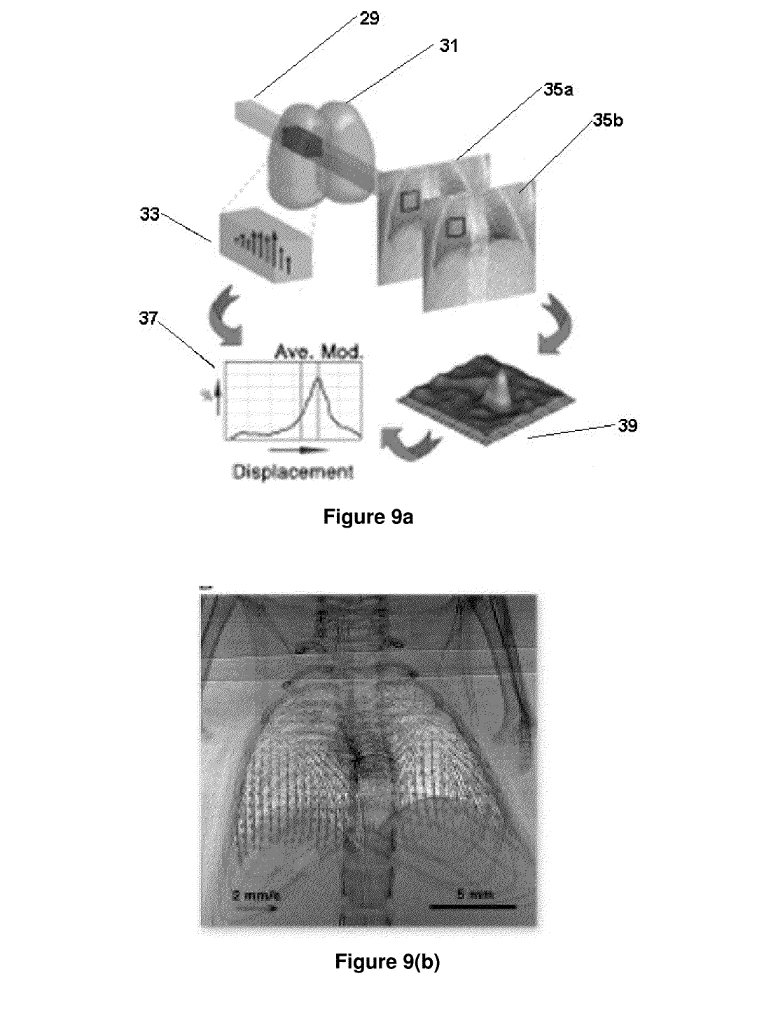

FIG. 9(a) shows the 3D nature of x-ray illumination and velocimetric cross-correlation analysis. Each 2D sampling region in the projection images represents a 3D volume for which a distribution of velocities may be present. The preferred parameter for the present invention is the modal velocity, which may significantly differ from the mean. FIG. 9(b) illustrates in vivo detection of lung tissue motion using instantaneous velocity of a healthy mouse lung ˜140 ms after the start of inspiration, shown as a vector field. Vectors are reduced in number (293 of 2640 displayed) for clarity. Vectors are coloured according to magnitude (from lowest; blue, to highest; red) of velocity. The complete time sequence of inspiration consists of 70 instantaneous vector fields (media), one of which is shown.

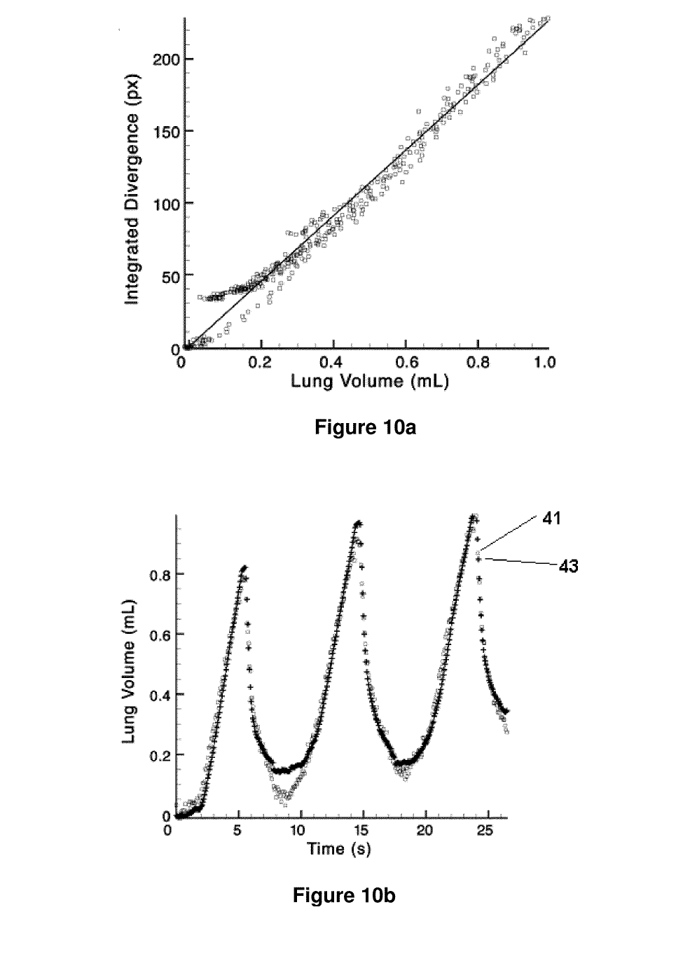

FIG. 10 shows the empirical relationship between lung divergence and tissue expansion. FIG. 10(a) is a scatter-plot of divergence (integrated throughout entire data series) and lung volume (measured by water plethysmography) of a rabbit measured from its first breath. The solid line indicates a line of best fit, with an R²=0.98 indicating excellent correlation between the data sets. FIG. 10(b) shows a time-series of lung volume (measured by water plethysmography) co-plotted with divergence (integrated throughout the entire data series and normalised by the co-efficient determined by the fit in FIG. 10(a).

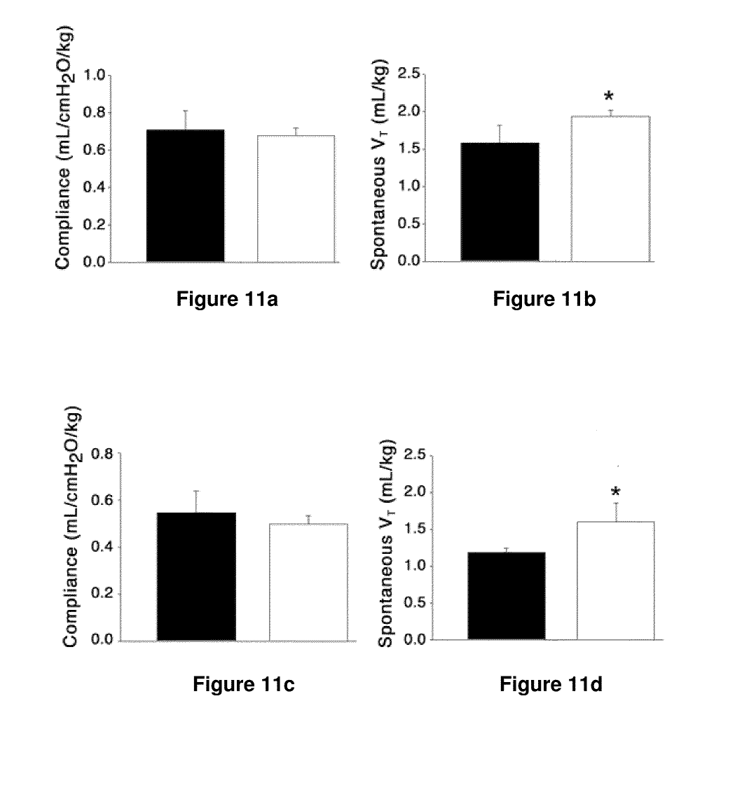

FIG. 11 shows physiological measures of lung pathology comparing the compliance for treated groups with controls (statistically insignificant) at 36 hours (FIG. 10(a)) and 6 days (FIG. 10(c)) after treatment. Comparisons of the spontaneous tidal volumes (V_{T}) at 36 hours (FIG. 10(b)) and 6 days after treatment (FIG. 10(d)). Tidal volumes in controls are significantly lower than treated groups but are non-specific and global in nature.

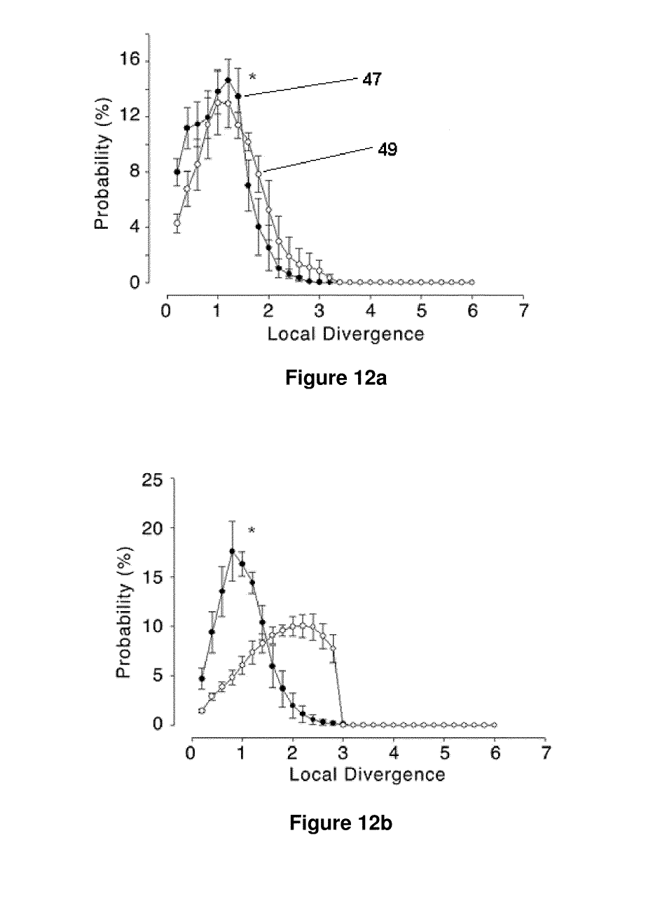

FIG. 12 shows velocimetric measures of lung pathology comparing controls with groups 36 hours and 6 days after bleomycin exposure. Frequency distribution of the divergence (normalised to the average of controls) is compared for treated groups (n=4) with controls (n=3). Data are normalised by the average of the controls. As shown by FIG. 12(a), at 36 hours post treatment, treated mice have 24% greater divergence on average and 14% of treated lungs show difference 2× the control average compared with less than 5% for control lungs. As shown by FIG. 12(b), at 6 days post treatment, treated mice have 76% greater divergence on average and 47% of treated lungs show differences 2× the control average compared with less than 4% for control lungs.

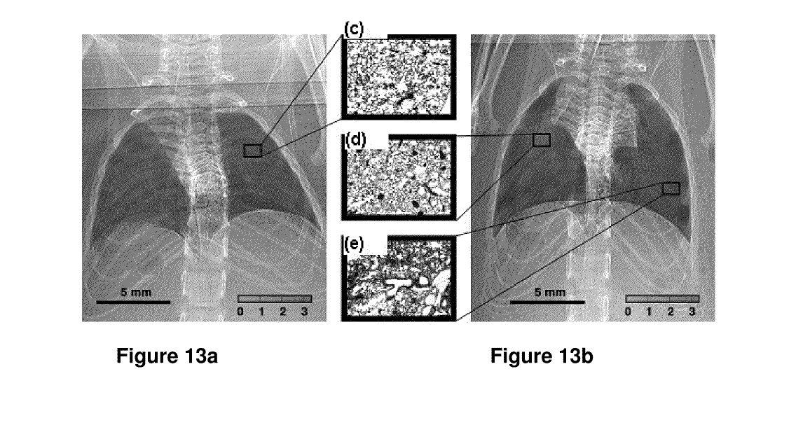

FIG. 13 shows regional divergence within the lung and matching histology. FIGS. 13(a) and 13(b) are colour maps of regional divergence determined using x-ray velocimetry for typical (a) control, and (b) bleomycin-treated mice (6 days after exposure). Data are normalised by the average divergence across the control group and colour maps generated using the same colour scale (see legend). The mice treated with bleomycin (b) have dramatic regional alterations in the pattern of divergence. Histological image (c) from lung imaged in (a) is typical of the control group. Histological images (FIGS. 13(d) and 13(e)) from lung imaged in (b) are typical of the pathological group 6 days after bleomycin treatment. Treated lungs show both regions of healthy tissue (d) and localised regions that are both hypercellular and endatemous (e).

### Experimental

The following non-limiting example illustrates how the combination of PCXI and velocimetry can produce quantitative measures of regional lung motion, which can be used to differentiate between normal and abnormal lung tissue. Furthermore the example illustrates that this technology is more sensitive and provides richer quantitative information for disease detection than other conventional measure such as global lung function tests and non-biased histological sampling.

Specifically, the present example illustrates the present invention when using single camera/2D imaging. Furthermore it illustrates the use of divergence as a measure of lung expansion. Despite the two-dimensional nature of imaging, the divergence measure is highly correlated to changes in lung volume. This measure was evaluated in a Bleomycin-induced lung injury model in immuno-deficient Balb/c nude mice that are known to have a reduced inflammatory response to bleomycin compared to other strains. Furthermore, mice are examined at 36 hours and 6 days after treatment to examine the early states of disease and determine whether disease progression can be detected.

### Materials and Methods

Protocol: Adolescent Balb/c nude male mice were exposed to bleomycin (20 mg/kg in 20 uL saline; n=8; Sigma-Aldrich, Australia) or saline (20 uL; n=6) by intranasal instillation and lung function was tested daily using whole body plethysmography. Mice were imaged at 36 h (n=4) or 6 days (n=4) after treatment. For imaging, mice were anaesthetized (pentobarbital; 15 mg/kg i.p.), muscle relaxed (Pancuronium 1 mg/kg i.m.), intubated and placed in a prewarmed (37° C.) water-filled plethysmograph. During imaging, mice were ventilated using a custom-designed ventilator at a peak inspiratory pressure of 20 cmH₂O and end expiratory pressure of 2 cmH₂O. Inspiration and expiration times were 2.5 s and 1.5 s respectively. Although this is significantly less than the normal ventilation rate for free-breathing mice, the imaging procedures only lasted for 5 breaths (20 s) and was not expected to result in hypoxia or any changes in lung mechanics for these somnolent mice. Following imaging, mice were killed (Pentobarbital; 100 mg/kg i.p.) and the lungs fixed (in 10% formalin) via the airways at a distending pressure of 20 cmH₂O. Paraffin-embedded sections (5 μm) were stained with Massons Trichrome and used for histological analysis. 5 fields of view were chosen at random from at least 3 randomly selected sections per mouse to measure the relative volume density of abnormal parenchymal lung regions. Then a subset analysis was performed to compare the relative tissue volume in normal and abnormal parenchymal regions using an unpaired T-test.

Phase-contrast X-ray imaging: Studies were conducted in experimental hutch 3 of BL20B2 at the Spring-8 synchrotron in Japan. The beamline consists of a bending magnet insertion device and Si-111 crystal monochromators, which generates a bright monochromatic X-ray beam. The X-ray beam transmits through the sample onto a scintillator, which converts the x-rays to visible light to be imaged by an optical detector system. Imaging was conducted at 25 keV with a sample-to-detector distance of 2 m. Images were acquired using an X-ray Converter (Hamamatsu, BM5) and an EMCCD (Hamamatsu, C9100-02) camera (FIG. 8(a)) with an effective pixel size of 19.0 um. Image acquisition occurred at 29 frames per second (an exposure time of 20 ms with a 14.5 ms delay between exposures, corresponding to 34.5 ms between the start of frame acquisitions) and was synchronized with ventilation to acquire 70 frames during the first part of inspiration and 30 frames during the first part of expiration for each breath. The mice were imaged in the upright position with all images acquired to obtain a frontal view of the entire thorax without the need for scanning or tiling. In all images (FIG. 9) the images are displayed without intensity inversion or laterally flipping and hence appear opposite in both regards in comparison to clinical x-ray images.

Velocimetry: The velocimetric analysis employed to measure lung motion is based on particle image velocimetry (PIV); this is an established technique for measuring differential fluid velocities, including blood flow. PIV determines the movement of particles from one image to the next, yielding information on both velocity and direction of particle movement. The basic concept is demonstrated in FIG. 9(a). Images are paired and discretised into small sub-regions and cross-correlations are performed between the sub-regions in consecutive images. The position of the maximum of the cross-correlation function determines the most common (modal) inter-frame displacement of the structures within each sub-region. Division of the displacement by the known inter-frame time yields the local modal velocity.

X-ray velocimetry has been utilised for the measurement of flow within channels for blood flow and has recently been adapted to 3D analysis. The high contrast intensity patterns produced by PCXI of the lung can be used instead of having to introduce exogenous particles as is the practice in conventional PIV. As a result, a comprehensive map displaying regional tissue velocities can be generated at all stages of the breathing cycle.

Whole animal motion was removed from image sequences by velocimetric analysis of upper vertebrae, followed by interpolation of images onto a static reference frame. Lungs were isolated from images by band-pass filtering using the appropriate image frequencies and regions containing the lungs were identified and masked. Following this pre-processing, velocimetric analysis was conducted for 5 consecutive inspirations using customised software and the date phase-average to produce data sets of 70 frames displaying the velocity vector fields throughout inspiration for each animal.

Divergence: At every time-point the spatial derivatives of the velocity fields can be evaluated and summed to form the two-dimensional divergence field. The spatial derivative distinguishes between bulk displacement of tissue and regional variations in tissue displacement, highlighting local differences in motion between regions. The local differences are directly related to local tissue expansion, and hence local variations in the divergence would be considered to be a measure of heterogeneity of tissue expansion and, by implication, tissue properties. For the sake of clarity of expression this projected divergence in motion will hereafter simply be referred to as the divergence. The total divergence over inspiration is the sum of the divergence between each pair of subsequent time points. As the data are integrated over the entire inspiration, total divergence is represented in a single map.

Statistics: Unpaired one-tailed t-tests were used to compare mean tidal volume and histological parameters. Two-way repeated measures ANOVA was used to determine differences in frequency distributions of divergence and time of divergence. Results were considered statistically significant at p<0.05. Values are reported as mean+/−SEM (unless stated otherwise).

### Results

Physiological analysis: Plethysmography was used to measure tidal volume (VT) during both spontaneous breathing and mechanical ventilation. Lung compliance was assessed only during mechanical ventilation, as airway pressure measurements were not available during spontaneous breathing. At 36 h after bleomycin treatment, the spontaneous VT was significantly increased from 1.4+/−0.1 mL/kg in saline-treated mice to 1.9+/−0.1 mL/kg in bleomycin-treated mice, whereas spontaneous breathing rates were reduced from 480+/−38 to 283+/−34 breaths/min. However, no significant differences in global lung compliance (0.68+/−0.04 vs 0.71+/−0.10 mL/cmH₂O/kg) could be detected during mechanical ventilation (FIG. 11). Similarly, at 6 days after bleomycin treatment, the spontaneous VT remained significantly elevated (1.2+/−0.1 vs 1.6+/−0.2 mL/kg) and the ventilation rate was reduced (440+/−13 vs 235+/−40 breaths/min), but no significant affect on global lung compliance (0.5+/−0.04 vs 0.55+/−0.05 mL/cmH₂O/kg) was detected during mechanical ventilation (FIGS. 11(c) & 11(d)).

Velocimetry: Determined using x-ray velocimetry, the velocity vectors define the timing and extent of regional lung motion throughout a breath. The vectors measured at mid-inspiration demonstrate that regional lung motion is very heterogeneous (FIG. 9) at this point in the breathing cycle.

Divergence—validation: To validate the divergence analysis, an existing, published, PCXI image data set was evaluated using the divergence analysis outlined herein. PCXI image were acquired as rabbit foetus lungs were slowly ventilated from their first breaths (in situ) inside a water plethysmograph—allowing concurrent measurements of lung air volume as it rises from zero.

The PCXI images were analysed for velocimetry and divergence. The divergence data was then integrated throughout the entire series rather than just each breath and compared to the plethysmograph data. A scatter-plot of the integrated divergence (FIG. 10(a)) plotted against the lung volume as measured by water plethysmograph shows strong correlation between the two quantities (R²=0.98). The gradient of the line of best-fit could be considered as a combination of the image magnification (pixel size) and an equivalent thickness of the lung since this gradient represents the empirically-determined conversion vector to convert between the change in area (in pixels) and a change in volume (mL). To demonstrate the direct relationship between these quantities, the divergence data has been normalised by the empirical thickness and plotted (FIG. 10(b)) with the lung volume as measured by water plethysmography against time. Despite some minor inaccuracies brought about by the imperfect sealing of the animal in the water plethysomgraph and the two-dimensional nature of the image data, the excellent agreement between these data demonstrates the direct link between divergence and tissue expansion and hence tissue mechanical properties.

Divergence—bleomycin treatment: Divergence data for bleomycin treated mice and controls were normalised by the average of controls and accumulated into frequency distribution curves, demonstrating that major difference (p<0.001) in regional lung motion can be detected between saline-treated and bleomycin-treated mice both 36 h and 6 days after treatment. Frequency distribution curves (FIG. 12) show that divergence, within individual regions of lung tissue, was on average 24% greater in bleomycin-treated mice compared to saline-treated controls at 36 h after treatment, despite having the same global VT. Moreover, in bleomycin treated mice at 36 h after treatment, 14% of lung regions showed local differences twice the mean value in saline-treated controls; less than 5% of lung regions showed this degree of divergence in control mice, indicating a three-fold increase between bleomycin-treated and control mice.

At 6 days after bleomycin treatment, the changes in lung motion were more enhanced than those detected at 36 h after treatment. Indeed, despite no changes in global lung compliance, x-ray velocimetry detected a highly significant shift in the frequency distribution curve towards greater divergence (FIG. 12(b)). On average, the divergence of individual lung regions was 76% greater in bleomycin-treated mice compared to saline-treated controls. Furthermore 47% of lung regions in treated mice show local differences in motion twice the mean value of saline-treated mice, while less than 4% of lung regions showed this degree of divergence in saline-treated mice. Hence there is nearly a 12 fold difference in divergence between bleomycin-treated mice and controls at 6 days after treatment. From these data, regional maps of divergence can be reconstructed and superimposed on the lung image to identify regions with abnormal motion (FIG. 13).

### Discussion

Although ventilators, plethysmographs and spirometers can measure many characteristics of lung function, those measures reflect the integrated average of the entire lung. As a result these techniques have limited ability to detect regional lung disease until it is sufficiently widespread to influence total lung function. In contrast, the present invention uses the capabilities of x-ray velocimetry to non-invasively detect breath-by-breath alterations in regional lung motion that occur even during the early stages of lung disease. Importantly, the velocimetric technique offers the advantages of detecting regional changes in lung function early, accurately and most importantly, in situ.

Superposition of the lung tissue velocity maps over the phase-contrast images acquire at mid-inspiration (140 ms after inspiration onset) clearly demonstrates the heterogeneity of lung motion (FIG. 9). Combining consecutive images reveals the changing dynamic of the velocity vector field during a breath and demonstrates that regional lung tissue motion is complex and non-linear. Indeed, regional velocities are most likely influenced by local characteristics of regional compliance, the compliance and motion of nearby tissue as well as the proximity to structure such as the diaphragm, heart and chest wall. For example, lung tissue near the diaphragm displayed significantly more motion than tissue near the apex of the lung (FIG. 9(b)) which is likely due to differences in compliance as well as motion and activity of the chest wall that is immediately adjacent to the lung tissue. To accommodate the large differential in normal motion across the lung, a functional measure was derived from the velocity fields to identify regions with abnormal motion potentially caused by disease. Specifically the local divergence was calculated, normalised to the average for all controls of reach treatment period (eg 36 hours or 6 days) so that differences could be detected.

The measure of divergence was derived from integration of the velocity vector field within each region over an entire breath. It is well understood that the divergence of a velocity field relates to the local expansion or contraction of the object, which in this case is lung tissue. As such the divergence accounts for normal variations in tissue motion (such as the increased motion near to the diaphragm compared to the apex) and converts heterogeneous patterns of tissue motion (displayed by normal tissue) to a homogenous pattern of divergence. However it is anticipated that heterogeneous regions of tissue properties (either resistance or compliance) will result in local variations in divergence.

However, lung tissue motion is three-dimensional and divergence measures are two-dimensional. Therefore the most correct interpretation of the measure of divergence is that it directly relates to local heterogeneity of lung tissue motion cause by differences in expansion. If all ventilation parameters, such as inflation times, pressures and gas flows are kept constant, their local heterogeneity in motion reflects differences in the mechanical response of lung tissue across the lung. This altered response must be due to either changes in the tissue mechanical properties, or a constriction/dilation of the airways leading to local alteration in resistance or compliance.

To test the ability of x-ray velocimetry and our subsequent analysis to detect abnormal lung motion, mice were exposed to bleomycin which resulted in progressive lung injury. Inhaled bleomycin is well characterised and commonly used experimental model of pulmonary fibrosis that begins with the initiation of an inflammatory cascade. Since Balb/c nude mice (an immuno-deficient strain) were utilised, it is not surprising that the pulmonary fibrotic response was reduced in these mice compared with reports in other strains. This is likely because inflammatory responses are reduced in these mice, although a recent study has also observed a similar reduced response in conventional Balb/c mice. In any event it is apparent that the induced pathological changes were insufficient to affect global lung compliance at the time points measured, as observed previously in conventional Balb/c mice. The mice were deliberately examined at 36 hours and 6 days after treatment which encompasses the early stages of disease pathogenesis when the induced changes can be detected histologically but not physiologically. Indeed a previous study has indicated that the maximum response to bleomycin occurs at 21 days for conventional Balb/c mice which is the only time that functional changes can be detected. However, it is clear from the histological analysis (FIG. 13) that the bleomycin treatment was sufficient to cause observable focal lesions within lung tissue.

The present velocimetric analysis in bleomycin-treated mice revealed highly significant changes in regional lung motion compared to saline-treated mice. Despite having similar tidal volumes and inflation pressures during mechanical ventilation (indicating no change in global compliance), bleomycin treatment increased divergence across the lung by 24% at 36 h and by 76% at 6 days. This highly sensitive measure yielded a three fold difference between the groups after only 36 hours of treatment and was increased further after 6 days of treatment.

It is apparent that it was not possible to detect a change in global lung compliance during mechanical ventilation at either 36 hours or 6 days after treatment, but that tidal volumes during spontaneous breathing were significantly increased by bleomycin treatment at both times (FIG. 11). The higher tidal volumes during spontaneous breathing were partially matched with lower ventilation rates resulting in minute ventilation (mL/min/kg) values that were slightly reduce by statistically similar in bleomycin and saline-treated mice.

In summary PCXI combined with velocimetry can measure regional lung motion and define the regional velocity changes at each stage of the respiratory cycle. Despite the large heterogeneity in normal motion across the lung, a detailed analysis of the velocity vectors can provide a very sensitive method for detecting abnormal motion caused by respiratory disease.

### Ventilator

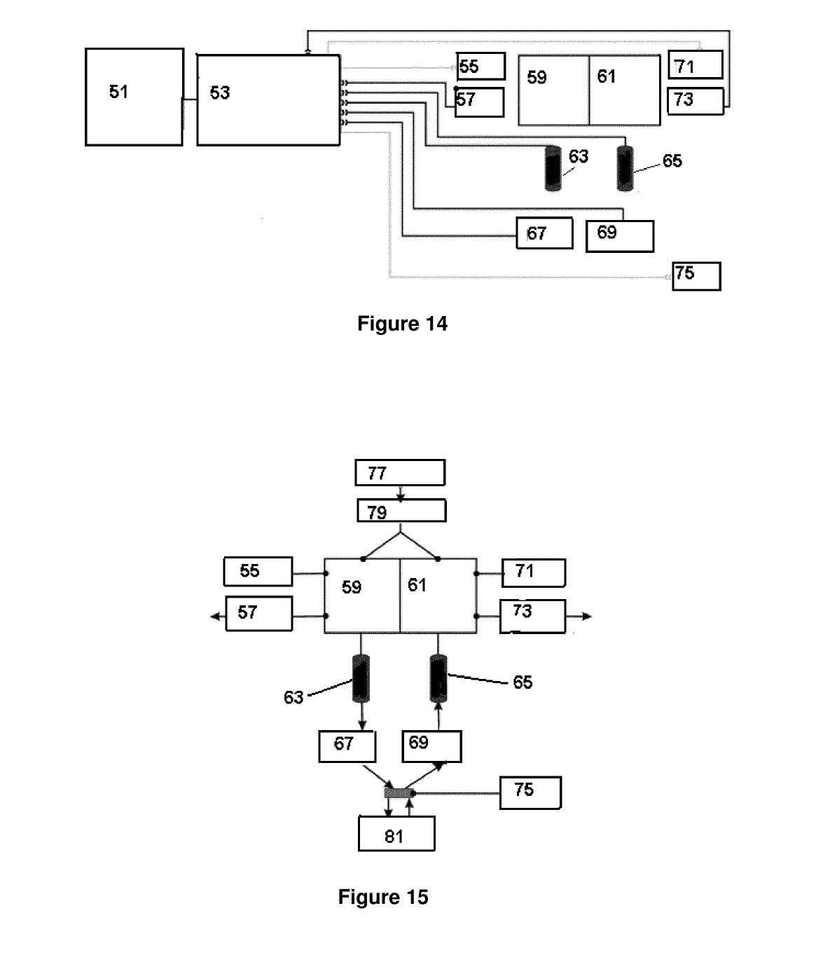

A time-cycled pressure-limited ventilator was developed, the ventilator operating using LabVIEW's Virtual Instrument (VI) controls to synchronise image acquisition with mechanical ventilation of small animals. A personal computer (PC) along with a data acquisition module (NI USB-6259) and National Instruments Lab VIEW software were used to control the ventilator, as illustrated in FIG. 14. Table 1 provides a detailed description of the ventilator components. The data acquisition system connects to the PC with a Universal Serial Bus (USB) cable and can handle up to 4 analog outputs, 80 analog inputs and 48 digital input/output channels at 1.25 MS/s. LabVIEW can simultaneously control multiple devices and readily interface with external hardware since many hardware drivers are included in the programming library. The main advantage of the virtual interface is that all parameters (eg air pressure and flow rate) can be controlled remotely with real-time display.

TABLE 1

| Component | Function | Specification |
|---|---|---|
| Data acquisition system (NI | To synchronise the PC with | 4 analog outputs, 80 analog |
| USB-6259) | the ventilator components | inputs (16-bit), 48 digital I/O, |
| | | 1.25 MS/s |
| LabVIEW software | To record outputs from and | Version 8.5 |
| | send inputs to the data | |
| | acquisition system | |
| Pump | To provide a continuous flow | |
| | of air | |
| Muffler | To dampen the pulsatile flow | 250 cm³ |
| | from the pump | |
| Pressure vessels (PIP and | To supply air of a fixed | Volume = approx. 100x larger |
| PEEP) | pressure to the lung during | than total volume of lung and |
| | inspiration and expiration | tubing |
| Solenoid valve (Cole- | To open and close during | Response time: 15 ms |
| Parmer, EW-01367-50) | inspiration and expiration | Max flow rate: 16 LPM |
| Stepping motor valves, | To maintain the pressure | Speed: 0-5 V DC Maximum flow |
| Aalborg, SMV40-S | inside the PIP and PEEP | rates: 1000 sL/min |
| | vessels and air flow to | Direction: TTL logic |
| | the lung | LED indicators |
| Differential pressure | To measure pressure within | LED range indication |
| transducer (DPT), Ashcroft | PIP and PEEP vessels and | Differential pressure ranges: 3- |
| DXLdp | the lung | 127 cm(H₂O) |

FIG. 15 depicts the manner in which air was cycled around the ventilator and delivered to the lung. Two pressure vessels, set at different pressures were used to control the PIP and PEEP. At the start of inspiration the inspiratory solenoid valve opened whilst the expiratory solenoid remained closed (FIGS. 14 and 15) for the entire set inspiratory time. Air from the PIP vessel flowed to the lung through the inspiratory solenoid via a variable restrictor valve; this allowed the air to flow into the lungs until the airway pressure reached the pressure of the PIP vessel. As the PIP and PEEP vessels were of sufficiently large volume (˜1 L/box), the volume change associated with opening and closing of the respiratory solenoids did not significantly influence the pressure within the vessels. This provided a high degree of stability with the applied PIP and PEEP pressures. A variable restrictor valve allowed almost infinite variability of the rate of gas flow into the lung from the pressure vessel, which in turn was controlled remotely via the virtual interface. As a result the inspiratory pressure wave form could be varied and the length of an inspiratory pressure plateau (as a proportion of inspiration time) could be set by regulating the inspiratory airflow independently of this PIP. Upon expiration the states of the respiratory solenoids are simultaneously flipped, allowing the lungs to deflate to the lower PEEP level for a preset period. During expiration, airflow from the lung into the PEEP box could also be regulated via a restrictor valve, as shown in FIG. 15. Both the inspiratory and expiratory times can be updated in software while the ventilator is operating. As a safety precaution the solenoids simultaneously closed when the ventilation sequence was terminated to prevent over-distension of the airways.

A ventilator of this type is capable of performing high frequency ventilation up to at least 33 Hz, which is well in excess of typical clinical usage of about 2 to 12 Hz.

While this invention has been described in connection with specific embodiments thereof, it will be understood that it is capable of further modification(s). This application is intended to cover any variations uses or adaptations of the invention following in general, the principles of the invention and including such departures from the present disclosure as come within known or customary practice within the art to which the invention pertains and as may be applied to the essential features hereinbefore set forth.

As the present invention may be embodied in several forms without departing from the spirit of the essential characteristics of the invention, it should be understood that the above described embodiments are not to limit the present invention unless otherwise specified, but rather should be construed broadly within the spirit and scope of the invention as defined in the appended claims. The described embodiments are to be considered in all respects as illustrative only and not restrictive.

Various modifications and equivalent arrangements are intended to be included within the spirit and scope of the invention and appended claims. Therefore, the specific embodiments are to be understood to be illustrative of the many ways in which the principles of the present invention may be practiced. In the following claims, means-plus-function clauses are intended to cover structures as performing the defined function and not only structural equivalents, but also equivalent structures. For example, although a nail and a screw may not be structural equivalents in that a nail employs a cylindrical surface to secure wooden parts together, whereas a screw employs a helical surface to secure wooden parts together, in the environment of fastening wooden parts, a nail and a screw are equivalent structures.

It should also be noted that where a flowchart is used herein to demonstrate various aspects of the invention, it should not be construed to limit the present invention to any particular logic flow or logic implementation. The described logic may be partitioned into different logic blocks (e.g., programs, modules, functions, or subroutines) without changing the overall results or otherwise departing from the true scope of the invention. Often, logic elements may be added, modified, omitted, performed in a different order, or implemented using different logic constructs (e.g., logic gates, looping primitives, conditional logic, and other logic constructs) without changing the overall results or otherwise departing from the true scope of the invention.

Various embodiments of the invention may be embodied in many different forms, including computer program logic for use with a processor (e.g., a microprocessor, microcontroller, digital signal processor, or general purpose computer and for that matter, any commercial processor may be used to implement the embodiments of the invention either as a single processor, serial or parallel set of processors in the system and, as such, examples of commercial processors include, but are not limited to Merced™, Pentium™, Pentium II™, Xeon™, Celeron™, Pentium Pro™, Efficeon™, Athlon™, AMD™ and the like), programmable logic for use with a programmable logic device (e.g., a Field Programmable Gate Array (FPGA) or other PLD), discrete components, integrated circuitry (e.g., an Application Specific Integrated Circuit (ASIC)), or any other means including any combination thereof. In an exemplary embodiment of the present invention, predominantly all of the communication between users and the server is implemented as a set of computer program instructions that is converted into a computer executable form, stored as such in a computer readable medium, and executed by a microprocessor under the control of an operating system.

Computer program logic implementing all or part of the functionality where described herein may be embodied in various forms, including a source code form, a computer executable form, and various intermediate forms (e.g., forms generated by an assembler, compiler, linker, or locator). Source code may include a series of computer program instructions implemented in any of various programming languages (e.g., an object code, an assembly language, or a high-level language such as Fortran, C, C++, JAVA, or HTML. Moreover, there are hundreds of available computer languages that may be used to implement embodiments of the invention, among the more common being Ada; Algol; APL; awk; Basic; C; C++; Conol; Delphi; Eiffel; Euphoria; Forth; Fortran; HTML; Icon; Java; Javascript; Lisp; Logo; Mathematica; MatLab; Miranda; Modula-2; Oberon; Pascal; Perl; PL/I; Prolog; Python; Rexx; SAS; Scheme; sed; Simula; Smalltalk; Snobol; SQL; Visual Basic; Visual C++; Linux and XML.) for use with various operating systems or operating environments. The source code may define and use various data structures and communication messages. The source code may be in a computer executable form (e.g., via an interpreter), or the source code may be converted (e.g., via a translator, assembler, or compiler) into a computer executable form.

The computer program may be fixed in any form (e.g., source code form, computer executable form, or an intermediate form) either permanently or transitorily in a tangible storage medium, such as a semiconductor memory device (e.g., a RAM, ROM, PROM, EEPROM, or Flash-Programmable RAM), a magnetic memory device (e.g., a diskette or fixed disk), an optical memory device (e.g., a CD-ROM or DVD-ROM), a PC card (e.g., PCMCIA card), or other memory device. The computer program may be fixed in any form in a signal that is transmittable to a computer using any of various communication technologies, including, but in no way limited to, analog technologies, digital technologies, optical technologies, wireless technologies (e.g., Bluetooth), networking technologies, and inter-networking technologies. The computer program may be distributed in any form as a removable storage medium with accompanying printed or electronic documentation (e.g., shrink wrapped software), preloaded with a computer system (e.g., on system ROM or fixed disk), or distributed from a server or electronic bulletin board over the communication system (e.g., the Internet or World Wide Web).

Hardware logic (including programmable logic for use with a programmable logic device) implementing all or part of the functionality where described herein may be designed using traditional manual methods, or may be designed, captured, simulated, or documented electronically using various tools, such as Computer Aided Design (CAD), a hardware description language (e.g., VHDL or AHDL), or a PLD programming language (e.g., PALASM, ABEL, or CUPL). Hardware logic may also be incorporated into display screens for implementing embodiments of the invention and which may be segmented display screens, analogue display screens, digital display screens, CRTs, LED screens, Plasma screens, liquid crystal diode screen, and the like.

Programmable logic may be fixed either permanently or transitorily in a tangible storage medium, such as a semiconductor memory device (e.g., a RAM, ROM, PROM, EEPROM, or Flash-Programmable RAM), a magnetic memory device (e.g., a diskette or fixed disk), an optical memory device (e.g., a CD-ROM or DVD-ROM), or other memory device. The programmable logic may be fixed in a signal that is transmittable to a computer using any of various communication technologies, including, but in no way limited to, analog technologies, digital technologies, optical technologies, wireless technologies (e.g., Bluetooth), networking technologies, and internetworking technologies. The programmable logic may be distributed as a removable storage medium with accompanying printed or electronic documentation (e.g., shrink wrapped software), preloaded with a computer system (e.g., on system ROM or fixed disk), or distributed from a server or electronic bulletin board over the communication system (e.g., the Internet or World Wide Web).

“Comprises/comprising” and “includes/including” when used in this specification is taken to specify the presence of stated features, integers, steps or components but does not preclude the presence or addition of one or more other features, integers, steps, components or groups thereof. Thus, unless the context clearly requires otherwise, throughout the description and the claims, the words ‘comprise’, ‘comprising’, ‘includes’, ‘including’ and the like are to be construed in an inclusive sense as opposed to an exclusive or exhaustive sense; that is to say, in the sense of “including, but not limited to”.

1. A method for dynamic investigation of a subject lung, the method comprising the steps of:
(i) imparting an oscillation to the lung at one or more forcing frequencies so as to elicit a lung response,
(ii) sensing the response of the lung simultaneously with the imparting of the oscillation to elicit a lung response,
(iii) choosing at least one parameter used in the sensing to define the lung motion associated with the lung response,
(iv) comparing one of the chosen parameters at each forcing frequency with the response at the forcing frequency in at least one region of the lung, and
(v) recording the comparison of step (iv).

2. A method according to claim 1 wherein the sensing comprises imaging the response.

3. A method according to claim 1 which comprises the additional step (vi) of comparing a parameter between different regions throughout the lung and creating a visual representation thereof.

4. A method according to claim 1 wherein the oscillation is externally applied oscillation chosen from the group comprising mechanical input oscillation, external chest wall oscillation, heart oscillation or combinations thereof.

5. A method according to claim 1 wherein the oscillation imparted to the lung is of a single frequency.

6. A method according to claim 1 wherein the oscillation imparted to the lung is of multiple frequencies applied simultaneously or one after the other.

7. A method according to claim 3 wherein the imaging is carried out in one dimension, two dimensions or three dimensions.

8. A method according to claim 3 wherein the imaging is carried out by a three dimensional method chosen from the group comprising magnetic resonance imaging, computer tomographic imaging, X-ray imaging or ultrasound imaging.

9. An apparatus for dynamic investigation of a subject lung using the method of the present invention, wherein the apparatus comprises:
a ventilator for delivering fluid pressure to the lung;
a means for imparting an oscillation to the lung at one or more forcing frequencies so as to elicit a lung response
a means of sensing the motion of the lung simultaneously with imparting of the oscillation to elicit a lung response;
a means for measuring at least one parameter used in sensing and associated with the lung response at the forcing frequency in at least one region of the lung;
processing means for comparing one of the sensing parameters at the forcing frequency with the response at the forcing frequency in at least one region of the lung;
a means for recording the results of the comparison for at least one region of the lung.

11. An apparatus according to claim 9 wherein the ventilator for delivering fluid pressure to the subject lung has:
a pump in operative connection with a first pressure vessel for control of the peak inspiratory pressure (PIP) of the subject, and
a housing for enclosure of the subject, the housing being in operative connection with the first pressure vessel.

12. An apparatus according to claim 11 wherein the ventilator can maintain stable and accurate pressure to the subject while an oscillation is delivered to the lung.

13. An apparatus according to claim 11 wherein the ventilator provides high frequency ventilation.

14. An apparatus according to claim 11 wherein the ventilator has at least two chambers.

15. An apparatus according to claim 11 wherein the ventilator additionally has;
a second pressure vessel for control of the positive end expiratory pressure (PEEP) of the subject, and
wherein the volumes of the first pressure vessel and the second pressure vessel are substantially greater than the volume of the lungs of the subject.

16. An apparatus according to claim 9 wherein the ventilator delivers stable pressure the lungs of the subject, using the steps of:
(1) enclosing the subject within a housing, the housing being in operative connection with a first pressure vessel held at a first (PIP) pressure and a second pressure vessel held at a second (PEEP) pressure, respectively,
(2) admitting air from the first pressure vessel into the housing, then
(3) admitting air from the housing into the second pressure vessel, and
(4) repeating steps (2) and (3) multiple times.

17. An apparatus according to claim 16 wherein the pressure within the first pressure vessel and the second pressure vessel does not change by more than ±10%.

18. A method according to claim 2 which comprises the additional step (vi) of comparing a parameter between different regions throughout the lung and creating a visual representation thereof.

19. A method according to claim 2 wherein the oscillation is externally applied oscillation chosen from the group comprising mechanical input oscillation, external chest wall oscillation, heart oscillation or combinations thereof.

20. A method according to claim 2 wherein the oscillation imparted to the lung is of a single frequency.

21. A method according to claim 2 wherein the oscillation imparted to the lung is of multiple frequencies applied simultaneously or one after the other.

22. A method according to claim 18 wherein the imaging is carried out in one dimension, two dimensions or three dimensions.

23. A method according to claim 18 wherein the imaging is carried out by a three dimensional method chosen from the group comprising magnetic resonance imaging, computer tomographic imaging, X-ray imaging or ultrasound imaging.
